(19) United States
(12) Patent Application Publication
(10) Pub. No.: US 2004/0043482 A1
(43) Pub. Date: Mar. 4, 2004

(54) Method of producing stem cell lines

(76) Inventor: Kye-Hyung Paik, Rancho Sante Fe, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/337,248

(22) Filed: Jan. 3, 2003

Related U.S. Application Data
(60) Provisional application No. 60/345,854, filed on Jan. 4, 2002.

(51) Int. Cl. C12N 5/08; C12N 5/06

(52) U.S. Cl. 435/325; 435/366

(57) ABSTRACT

A method of producing a cell line from a pluripotent cell by isolating a portion of a feeder tissue from a first animal, maintaining the feeder tissue in contact with culture medium, contacting and incubating a pluripotent cell from a second animal together with the feeder tissue in the culture medium, and recovering a cell line wherein the cell line is derived from a pluripotent cell. This method can be used to prevent pluripotent cells from differentiating or aging in an in vitro setting by using fibroblast-rich feeder tissue slices. This method can also be used to produce differentiated cell lines suitable for use in transplantation.

### RELATED APPLICATION

This application claims priority from U.S. Provisional Patent Application No. 60/345,854, filed on Jan. 4, 2002, and entitled METHOD OF PRODUCING STEM CELL LINES, the disclosure of which is hereby incorporated by reference in its entirety.

### BACKGROUND OF THE INVENTION

1. Field of the Invention

The present invention relates to a method of producing differentiated cell lines from pluripotent stem cells using feeder tissues.

2. Description of the Related Art

The first human pluripotent embryonic stem cell line was established and named H1, H7, H9, H13, and H14 by the Thomson group at the University of Wisconsin in the United States. See Thomson, J. A. et al (1998) Embryonic stem cell lines derived from human blastocysts, Science 282:1145-1147. A few years later, Dr. Pera and his colleagues reported having established human embryonic stem cell lines (“ES cell lines”), which were named HES-1 and HES-2, from human blastocysts. See Reubinoff, B. E. et al. (2000) Embryonic stem cell lines from human blastocysts: somatic differentiation in vitro, Nat. Biotechnol. 18:399-404. Both human ES cell lines were derived from embryos produced by in vitro fertilization for clinical purposes. These two research groups developed the ES cell lines using similar procedures.

Feeder cell layers were used to provide a microenvironment (or niche) to prevent stem cells from differentiating along their natural course. Examples of feeder cells are: (1) irradiation-inactivated mouse embryonic fibroblasts; (2) mitotically (mitomycin C) inactivated mouse embryonic fibroblasts; and (3) irradiation-inactivated STO fibroblast feeder layers. See Thomson, J. A. et al, (1998); Reubinoff B. E. et. al. (2000); and Shamblott, M. J. et al. (1998) Derivation of pluripotent stem cells from cultured human primordial germ cells; Proc. Natl. Acad. Sci. U.S.A. 95:13726-13731.

The second method of providing a proficient microenvironment that will allow stem cells to develop into differentiated adult cells used fresh tissue maintained in a culture system. Feeder tissues provide the stem cells with external signals such as secretion of factors and cell-to-cell interactions mediated by integral membrane proteins. See Watt F. M. and Hogan L. M. (2000) Out of Eden: stem cells and their niches, Science 287:1427-1430. In light of the fact that secretion factors and direct cell-to-cell interactions control in vitro survival, proliferation, and differentiation of the stem cells, an ideal environment should consist of healthy feeder tissues with normal microstructures and functions.

Human development differs dramatically from mouse development in the timing of embryonic genome expression. See Braude, P. et al. (1988). Conventionally, feeder cells are taken from cell layers originating from mouse embryos. It is unknown if human ES cells propagated onto mouse feeder cells are modified in undesired ways. Human ES cells take up a variety of soluble factors secreted from mouse feeder cells as well as directly contact the feeder cell. The mouse feeder cells influence the identity of pluripotent human ES cells in ways which may be deleterious for human therapy. Furthermore, adult tissues or cells that differentiated from human ES cells may be unsuitable for human clinical trials because it may be impossible to separate the human cells from the mouse feeder cells or their debris. The mouse cells or debris contamination may bring about undesirable consequences.

Human gene expression first occurs in the formation, structure and function of the fetal membranes and placenta and in the formation of an embryonic disc. See Luckett, W. P. (1978) Origin and differentiation of the yolk sac and extraembryonic mesoderm in primate human and rhesus monkey embryos, Am. J Anat. 152:59-98; O'Rahilly, R. and Muller, F. (1987) Developmental stages in human embryos, Caregie Institution of Washington; Thomson, J. A. and Odorico, J. S. (2000) Human embryonic stem cell and embryonic germ lines, Trends in Biotechnol. 18:53-57). This differentiation occurs approximately between the four-cell and eight-cell stages of preimplantation development. See Benirschke, K. and Kaufmann, P. (1990) Pathology of the human placenta, Nature 332:459-461.

### SUMMARY OF THE INVENTION

According to the invention, there is provided a method of producing a cell line from a pluripotent cell wherein a portion of an organ or a tissue is isolated from a first animal to be used as a feeder tissue, the feeder tissue is maintained in contact with a culture medium, a pluripotent cell from a second animal is contacted with the feeder tissue, the pluripotent cell is incubated with the feeder tissue, and a cell line derived from the pluripotent cell is recovered.

In one embodiment of the invention, the first and second animal are from the same species. The first and second animal can also be from the same individual. Alternatively, the first and second animal can be from different species.

In one aspect of the invention, the pluripotent cell comprises a totipotent cell, stem cell, embryonic germ cell, multipotent stem cells of brain, liver, heart, pancreas, blood, or other tissue in the body. In another aspect of the invention, the pluripotent cell is isolated from vertebrates or invertebrates. In yet another aspect of the invention, the feeder tissue is isolated from animal organs. In another aspect of the invention, the feeder tissue is isolated from vertebrates or invertebrates. In still another aspect of the invention, the feeder tissue can be replaced with fresh feeder tissue at any time.

According to the invention there is provided a method of a preventing a stem cell from differentiating or aging in vitro wherein a portion of a fibroblast-rich organ or tissue is isolated from a first animal to be used as a fibroblast rich feeder tissue, the fibroblast rich feeder tissue is maintained in contact with a culture medium, a pluripotent cell from a second animal is contacted with the fibroblast rich feeder tissue, the pluripotent cell is incubated with the fibroblast rich feeder tissue, and a cell line derived from the pluripotent cell is recovered.

In one aspect of the invention, the pluripotent cell comprises a totipotent cell, stem cell, embryonic germ cell, multipotent stem cells of brain, liver, heart, pancreas, blood, or other tissue in the body. In another aspect of the invention, the pluripotent cell is isolated from vertebrates or invertebrates. In yet another aspect of the invention, the fibroblast rich feeder tissue is isolated from fresh granulation tissue in chronic inflammatory tissue or fibrosarcoma. In another aspect of the invention, the fibroblast rich feeder tissue is isolated from vertebrates or invertebrates. In still another aspect of the invention, the feeder tissue can be replaced with fresh feeder tissue at any time.

According to the invention there is provided a method for producing a differentiated cell line suitable for use in transplantation wherein a portion of a targeted organ or tissue is selected from a first animal to be used as a feeder tissue, the feeder tissue is maintained in contact with a culture medium, a pluripotent cell from a second animal is contacted with the feeder tissue, the pluripotent cell is incubated with the feeder tissue, and a differentiated cell line derived from the pluripotent cell is recovered.

In one aspect of the invention, the feeder tissue is cultured in vitro. In another aspect of the invention, the pluripotent cell is isolated from a human. In another aspect of the invention, the targeted organ or tissue to be used as a feeder tissue comprises a dopaminergic neuron. In another aspect of the invention, the differentiated cell line is suitable for use in the transplantation of nerve cells for treatment of neurodegenerative diseases. In yet another aspect of the invention, such neurodegenerative diseases are Alzheimer's disease or Parkinsonism.

In another aspect of the invention, the cell line produced from the human pluripotent cell is a human nerve cell. In still another aspect of the invention, the targeted organ or tissue to be used as a feeder tissue comprises cardiac muscle. In another aspect of the invention, the differentiated cell line is suitable for use in transplantation of cardiac muscle for treatment of myocardial infarction. In still another aspect of the invention, the cell line produced from the human pluripotent cell is a human heart cell. In another aspect of the invention, the targeted organ or tissue to be used as a feeder tissue comprises liver or hepatic tissue. In still another aspect of the invention, the differentiated cell line is suitable for use in transplantation of liver or hepatic tissue for treatment of end stage liver disease. In another aspect of the invention, the cell line produced from the human pluripotent cell is a human liver cell.

### DETAILED DESCRIPTION OF THE PREFERRED EMBODIMENT

Conventional pluripotent cell culture technology does not readily allow production of natural cell lines. Producing natural cell lines, however, is necessary for the progression of human transplantation techniques. Cell lines can now be produced from pluripotent cells cultured with an isolated portion of an organ or tissue, called a feeder tissue. The feeder tissue provides a more natural environment upon which pluripotent cells may develop and differentiate into their natural adult cell state.

A need also exists to at least diminish if not stop pluripotent cells from differentiating or aging in an in vitro setting. To satisfy this need, however, are fibroblast-rich feeder tissues, which provide an environment that interrupts the normal tissue regeneration from progenitor cells. Slices of primate brain tissue can be cultured with human pluripotent cells and maintained in an automated dynamic culture system. Fresh brain tissue can be isolated from a variety of vertebrates and invertebrates using standard surgical procedures know to those of skill in the art.

A human feeder cell layer would offer a better microenvironment, than a mouse feeder cell, for maintaining nutritionally healthy and undifferentiated human ES cells. Human feeder cell layers may be required in order to provide an optimal niche in which pluripotent cells can develop. The use of feeder cell layers derived from human tissues has never been reported. It has been found that a dynamic organ culture system using human tissue slices maintains healthier cells that possess a higher concentration of mitochondrial organelles. Since tissue slices more closely resemble normal tissues, rather than cell lines cultured on a plastic dish, healthier tissue slices prevail as better feeder cell layers for in vitro stem cell development.

The method of producing a cell line from a pluripotent cell is described herein. This method includes isolating a portion of an organ or tissue from a first animal to be used as a feeder tissue. The organ or tissue is preferably sliced into approximately a 2 square centimeter portion, approximately 260 micrometers thick, to be used as the feeder tissue. An organ or a tissue from an animal can be used as the feeder tissue. The isolated organ or tissue slice, to be used as the feeder tissue, is taken from a first animal. This animal can be a vertebrate or an invertebrate species.

The feeder tissue is maintained in contact with a culture medium. The feeder tissue may grow, increasing in size as a result of accretion of tissue similar to that originally present, or the feeder tissue may simply maintain its original size and consistency. The culture medium used to maintain the feeder tissue is preferably Modified Waymouth's MB 752/1 culture medium at pH 7.0. The feeder tissue was cultured at approximately 37° C. under approximately 1.6 to 2 atmospheres of pressure. The feeder tissue was exposed to a gas mixture of 5% CO₂ and 95% O₂ which was exchanged at intervals of about 2.5 minutes. The feeder tissue was immersed into the culture medium about 4.5 times per minute by rotating the culture tube.

A pluripotent cell from a second animal was placed in contact with the feeder tissue. The pluripotent cell, as described herein, is a cell that possesses the power of developing or acting in any one of several possible ways, such as by affecting more than one organ or tissue. Totipotent cells are cells that have the ability to differentiate along any line or into any type of cell. Herein, the definition of pluripotent cell includes totipotent cells, stem cells, embryonic germ cells, multipotent stem cells, neurons, hepatocytes, myocardium, Beta islet cell of pancreas, and endothelium cells, as well as any other cell with the potential to differentiate along more than one differentiation pathway. The pluripotent cell was taken from a second animal. This animal can be a vertebrate or an invertebrate.

The present invention allows for cross-species combination of pluripotent cells and feeder tissues. The first animal, from which the feeder tissue is taken and the second animal, from which the pluripotent cell is taken, can be from the same species or different species. In fact, the feeder tissue and the pluripotent cell source can be taken from the same individual. This is especially important for providing functional neuron cells for the human brain. Normally, such neurons cannot be readily used as feeder tissue. It will be understood by those of skill in the art that feeder tissues can be harvested from, for example, a cardiac muscle, a liver, a skin, a spleen, a pancreas, bone marrow, a striated muscle, a bladder, a kidney, a reproductive organ, a vein, an artery, a hair sample, a mucous membrane, an olfactory membrane, an oral membrane, or a nasopharyngeal membrane, an intestinal membrane, a mammary gland, a lung, a prostrate, an optical tissue, a stomach, a fibroblast-rich tissue, or the like. Under such conditions, cell lines from a cardiac muscle cell, a hepatocyte, a keratinocyte, a Beta cell of pancreatic islet, a blood cell, a stem cell, or the like could be produced from the pluripotent stem cell.

The invention may be better understood by way of the following examples which are representative of the preferred embodiments, but which are not to be construed as limiting the scope of the invention.

### EXAMPLE 1

### Brain Cell Production Using Human Stem Cells and Primate Brain Feeder Tissue

Fresh brain tissue is isolated from a baboon using standard surgical procedures. The tissue is sliced into approximately 2 cm² pieces of about 260 μm thickness. The primate brain feeder tissue is incubated in culture medium containing human stem cells. In this system, the primate feeder tissue is cultured in a porous container placed inside a culture tube which is rotated to permit the tissue to be periodically immersed in the medium. Gas exchange within the culture tube occurs at regular intervals by introducing a gas mixture into the culture tube. The culture system is maintained at a constant temperature of 37° C. by placing it in an incubator.

The feeder tissue is maintained in medium consisting of Dulbeco's modified Eagle's medium (DMEM; no pyruvate, high glucose, Gibco-BRL) supplemented with 20% fetal bovine serum (Hyclone), 1-2 mM glutamine, 0.1 mM 2-mercaptoethanol (Sigma), 1% nonessential amino acid stock (Gibco-BRL), and with or without antibiotics at pH 7.0, under 1.6 to 2 atm of a gas mixture of 5% CO₂ and 95% O₂. See Thomson, J. A. et. al, 1998; Reubinoff, B. E. et al, 2000. Those skilled in the art will appreciate that other medium and gas mixtures can be equivalently used.

The incubation of stem cells and feeder tissues is generally from about 1 to 72 hours, preferably about 24 hours. Neuron-like clumps are removed by mechanical dissociation with a micropipette or by exposure to dispase (10 mg/ml, Sigma). The stem cells are then cultured with the feeder tissue in fresh medium. Cultured cells are examined to detect the specific cell markers for each cell as well as morphological studies including immunohistochemistry.

### EXAMPLE 2

### Producing Human Es Cell Line

The inner cell mass of human blastocysts are isolated by immunosurgery as described previously. See Solter D. and Knowles B., 1975. Immunosurgery is accomplished using anti-human serum followed by exposure to guinea pig complement. See id. The ICM is then plated on irradiated Thomson or Pera-mouse embryonic fibroblast medium containing mitomycin C. The culture medium used in this technique consists of Dulbeco's modified Eagle's medium (DMEM; no pyruvate, high glucose, Gibco-BRL) supplemented with 20% fetal bovine serum (Hyclone), 1-2 mM glutamine, 0.1 mM 2-mercaptoethanol (Sigma), 1% nonessential amino acid stock (Gibco-BRL), and with or without antibiotics. See Thomson, J. A. et al., (1998); Reubinoff, B. E. et al., (2000).

After 9 to 15 days (Thomson's) or 6 to 8 days (Pera's), ICM-like clumps are removed by mechanical dissociation with a micropipette or by exposure to dispase (10 mg/ml, Sigma). The ICM-like clumps are then replated on the same feeder cell layer and fresh medium is added. When Pera ES cell lines are used, human recombinant leukemia inhibitory factor (hLIF, from AMRAD in Melbourne, Australia) is supplemented in the growth medium at 2,000 units/ml during the isolation and early stages of cultivation.

A frozen fibroma tissue, sliced into about 2 cm² pieces and about 260 μm thickness, is thawed. Human stem cells and fibroma slices are maintained in an automated dynamic culture system. The tissue slices are cultured in a porous container placed inside a culture tube that is continuously rotated to permit periodic immersion of the tissue into the medium. Gas exchange within the culture tube occurs at regular intervals in which a gas mixture is introduced into the culture tube. The fibroma tissue and the stem cells are cultured at 37° C. in culture medium consisting of Dulbeco's modified Eagle's medium (DMEM; no pyruvate, high glucose, Gibco-BRL) supplemented with 20% fetal bovine serum (Hyclone), 1-2 mM glutamine, 0.1 mM 2-mercaptoethanol (Sigma), 1% nonessential amino acid stock (Gibco-BRL), and with or without antibiotics (Thomson, J. A. et. al, 1998) (Reubinoff, B. E. et al, 2000) at pH 7.0, under 1.6 to 2 atm of a gas mixture of 5% CO₂ and 95% O₂ although those skilled in the art will appreciate that other medium and gas mixtures can be equivalently used.

The culture system is maintained at a constant temperature of 37° C. by placing it in an incubator. Incubation of stem cells and feeder tissues is generally from about 1 to 72 hours. Inner cell mass-like clumps are removed by mechanical dissociation with a micropipette or by exposure to dispase (10 mg/ml, Sigma) followed by being cultured with the other feeder tissue in fresh medium.

The samples of cells from the culture system are examined for specific markers of stem cells with well known arts in this field. The markers specific for stem cells are as follows; Oct-4 transcription factor expression; high level of telomerase activity; high ratio of nucleus to cytoplasm; alkaline phosphatase activity; prominent nucleoli; absence of SSEA-1 (stage-specific embryonic antigen-1) expression; moderate expression of SSEA-3; high-level expression of SSEA-4; expression of high molecular weight glycoprotein TRA-1-60; and expression of high molecular weight glycoprotein TRA-1-81.

Another means for producing cell lines from stem cells using tissue feeders is to inject pluripotent cells into severe combined immunodeficient (SCID)-beige mice. After observing the production of a teratoma, including endoderm, ectoderm, and mesoderm, the teratoma is cultured. ES colony morphology is characterized by flat and distinct borders between individual cells.

### EXAMPLE 3

### Nerve Cell Production Using Human Es Cells and Baboon Brain Feeder Tissue

Nerve feeder tissue and stem cells are cultured at 37° C. in culture medium consisting of Dulbeco's modified Eagle's medium (DMEM; no pyruvate, high glucose, Gibco-BRL) supplemented with 20% fetal bovine serum (Hyclone), 1-2 mM glutamine, 0.1 mM 2-mercaptoethanol (Sigma), 1% nonessential amino acid stock (Gibco-BRL), and with or without antibiotics at pH 7.0, under 1.6 to 2 atm of a gas mixture of 5% CO₂ and 95% O₂, although those skilled in the art will appreciate that other medium and gas mixtures can be equivalently used. See Thomson, J. A. et. al, 1998; Reubinoff, B. E. et al, 2000. The culture system is maintained at a constant temperature of 37° C. by placing it in an incubator.

Incubation of stem cells and feeder tissues is generally from about 1 to 72 hours, neuron-like clumps are removed by mechanical dissociation with a micropipette or by exposure to dispase (10 mg/ml, Sigma) followed by being cultured with the other feeder tissue in fresh medium. The samples of cells from the culture system are examined for specific markers of nerve cells with well known arts in this field. The markers specific for stem cells are the same as those mentioned above in Example 1.

### EXAMPLE 4

### Hepatocyte Production Using Frozen Human Liver Feeder Tissue and Baboon Stem Cells

Stem cell lines are established from baboon blastocyst using the technique described in Example 1. A frozen liver sliced into approximately 2 cm² pieces of about 60μ thickness was thawed. Baboon stem cells and human liver feeder tissues are maintained in an automated dynamic culture system. The feeder tissue is cultured in a porous container placed inside a culture tube which is continuously rotated in order to permit the tissue to be periodically immersed in the tissue culture medium as the culture tube is rotated. Gas exchange within the culture tube occurs at regular intervals in which a gas mixture is introduced into the culture tube.

The liver feeder tissue and the stem cells are cultured at 37° C. in culture medium consisting of Dulbeco's modified Eagle's medium (DMEM; no pyruvate, high glucose, Gibco-BRL) supplemented with 20% fetal bovine serum (Hyclone), 1-2 mM glutamine, 0.1 mM 2-mercaptoethanol (Sigma), 1% nonessential amino acid stock (Gibco-BRL), and with or without antibiotics at pH 7.0, under 1.6 to 2 atm of a gas mixture of 5% CO₂ and 95% O₂, although those skilled in the art will appreciate that other medium and gas mixtures can be equivalently used. See Thomson, J. A. et. al, 1998; Reubinoff, B. E. et al, 2000. The culture system is maintained at a constant temperature of 37° C. by placing it in an incubator.

Incubation of stem cells and feeder tissue is generally from about 1 to 72 hours, inner cell mass-like clumps are removed by mechanical dissociation with a micropipette or by exposure to dispase (10 mg/ml, Sigma) followed by being cultured with the other feeder tissue in fresh medium. The samples of cells from the culture system are examined with well known arts in this field.

### EXAMPLE 5

### Cardiac Muscle Cell Production Using Human Stem Cells and Human Heart Feeder Tissue

The inner cell mass of human blastocysts are isolated by immunosurgery as described previously. See Solter D. and Knowles B., 1975. A frozen myocardium sliced from a surgical specimen into approximately 2 cm² pieces of about 260 μm thickness is thawed. Human stem cells and myocardium slices are maintained in an automated dynamic culture system. The feeder tissue is cultured in a porous container placed inside of a culture tube which is rotated to permit the tissue to be periodically immersed in the tissue culture medium. Gas exchange within the culture tube occurs at regular intervals in which a gas mixture is introduced into the culture tube.

The myocardium feeder tissue and the stem cells are cultured at 37° C. in culture medium consisting of Dulbeco's modified Eagle's medium (DMEM; no pyruvate, high glucose, Gibco-BRL) supplemented with 20% fetal bovine serum (Hyclone), 1-2 mM glutamine, 0.1 mM 2-mercaptoethanol (Sigma), 1% nonessential amino acid stock (Gibco-BRL), and with or without antibiotics at pH 7.0, under 1.6 to 2 atm of a gas mixture of 5% CO₂ and 95% O₂, although those skilled in the art will appreciate that other medium and gas mixtures can be equivalently used. See Thomson, J. A. et. al, 1998; Reubinoff, B. E. et al, 2000. The culture system is maintained at a constant temperature of 37° C. by placing it in an incubator. Incubation of stem cells and feeder tissues is generally from about 1 to 72 hours, myocyte-like clumps are removed by mechanical dissociation with a micropipette or by exposure to dispase (10 mg/ml, Sigma) followed by being cultured with the other fresh feeder tissue in fresh medium. The samples of cells from the culture system are examined with well known arts in this field.

### EXAMPLE 6

### Skin Cell Production Using Human Pluripotent Cells and Human Epidermal Feeder Tissue

Epidermal tissue is isolated from a human and sliced into approximately 2 cm² pieces of about 260 μm thickness by well known methods in this field. Human pluripotent cells and human epidermal feeder tissue are maintained in an automated dynamic culture system. The epidermal feeder tissue is cultured in a porous container placed inside of a culture tube. The culture medium used herein may be comprised of fetal bovine serum, sodium bicarbonate, D-glucose, and crystalline bovine zinc insulin. The medium may further contain water, preferably distilled water. The culture medium may also contain one or more antibiotics, preferably penicillin or streptomycin. The feeder tissue is periodically immersed into the culture medium by rotating the culture tube to permit periodic immersion of the tissue into the culture medium. Gas exchange within the culture tube is timed to occur at regular intervals in which a gas mixture is introduced. The gas exchange may be approximately 5% CO₂ and 95% O₂ under approximately 1.6 to 2 atmospheres of pressure. See Thomson, J. A. et. al, 1998; Reubinoff, B. E. et al, 2000.

Incubation of the human pluripotent cells and the human epidermal feeder tissue is generally from about 1 to 72 hours. At any time during the incubation the feeder tissue may be replaced by fresh feeder tissue in fresh medium. The samples of cells from the culture system are examined for specific markers of epidermal cells by well known arts in this field.

### EXAMPLE 7

### Kidney Cell Production Using Human Pluripotent Cells and Swine Kidney Feeder Tissue

Swine kidney tissue is isolated by immunosurgery by methods well known in the art. A frozen swine kidney feeder tissue is sliced from a surgical specimen into approximately 2 cm² pieces of about 260μ thickness is thawed. Human pluripotent cells and swine kidney feeder tissues are maintained in an automated dynamic culture system. The swine kidney feeder tissues are cultured in a porous container and placed in a culture tube which is rotated to permit the tissue to be periodically immersed in the tissue culture medium. Gas exchange within the culture tube occurs at regular intervals in which a gas mixture is introduced into the culture tube.

The swine kidney tissues and human pluripotent cells are cultured as previously described in Example 6. The culture system is maintained at a constant temperature of 37° C. by placing it in an incubator. Incubation of human pluripotent cells and swine kidney feeder tissues is generally from about 1 to 72 hours. This incubation step generally lasts no longer than about one week. The samples of cells from the culture system were examined by well known methods in this field. The human kidney cells may be recovered by immunosurgery. See id.

### EXAMPLE 8

### Corneal Cell Production Using Human Pluripotent Cells and Human Corneal Feeder Tissue

Human corneal tissue slice and human pluripotent cells are cultured at 37° C. in culture medium consisting of Dulbeco's modified Eagle's medium (DMEM; no pyruvate, high glucose, Gibco-BRL) supplemented with 20% fetal bovine serum (Hyclone), 1-2 mM glutamine, 0.1 mM 2-mercaptoethanol (Sigma), 1% nonessential amino acid stock (Gibco-BRL), and with or without antibiotics at pH 7.0, under 1.6 to 2 atm of a gas mixture of 5% CO₂ and 95% O₂ although those skilled in the art will appreciate that other medium and gas mixtures can be equivalently used. See Thomson, J. A. et. al, 1998; Reubinoff, B. E. et al, 2000. The culture system is maintained at a constant temperature of 37° C. by placing it in an incubator.

Incubation of pluripotent cells and corneal feeder tissue is generally from about 1 to 72 hours. The corneal feeder tissue can be replaced at any time during incubation with fresh corneal feeder tissue and fresh medium. The samples of cells from the culture system are examined for specific markers of corneal cells by well known arts in this field.

What is claimed is:

1. A method of producing a cell line from a pluripotent cell comprising:
isolating a tissue from a first animal to be used as a feeder tissue;
maintaining the feeder tissue in contact with a culture medium;
contacting a pluripotent cell from a second animal with the feeder tissue;
incubating the pluripotent cell together with the feeder tissue in the culture medium; and
recovering a cell line derived from the pluripotent cell.

2. The method of claim 1, wherein the first animal and the second animal are from the same species.

3. The method of claim 2, wherein the first animal and the second animal are the same individual.

4. The method of claim 1, wherein the first animal and the second animal are from different species.

5. The method of claim 1, wherein the pluripotent cell is selected from the group consisting of a totipotent cell, a stem cell, an embryonic germ cell, and a multipotent stem cell.

6. The method of claim 5, wherein tissue from which the pluripotent cell is selected from the group consisting of a brain tissue, a liver tissue, a heart tissue, a pancreas tissue, and a blood tissue.

7. The method of claim 1, wherein the pluripotent cell is isolated from a vertebrate or an invertebrate.

8. The method of claim 1, wherein the feeder tissue is isolated from an animal.

9. The method of claim 1, wherein the feeder tissue is isolated from a vertebrate or an invertebrate.

10. The method of claim 1, further comprising replacing the feeder tissue with fresh feeder tissue.

11. A method of a preventing a stem cell from differentiating or aging in vitro, comprising:
isolating a fibroblast-rich tissue from a first animal to be used as a fibroblast rich feeder tissue;
maintaining the fibroblast rich feeder tissue in contact with a culture medium;
contacting a pluripotent cell from a second animal with the fibroblast rich feeder tissue;
incubating the pluripotent cell with the fibroblast rich feeder tissue in the culture medium; and
recovering a cell line derived from the pluripotent cell;
wherein the recovered cell line is prevented from differentiating or aging in vitro.

12. The method of claim 11, wherein the fibroblast rich feeder tissue is isolated from a fresh granulation tissue in a chronic inflammatory tissue or a fibrosarcoma.

13. The method of claim 11, wherein the fibroblast rich feeder tissue is isolated from a vertebrate or an invertebrate.

14. The method of claim 11, further comprising replacing the feeder tissue with fresh feeder tissue.

15. A method for producing a differentiated cell line suitable for use in transplantation comprising:
selecting a portion of a tissue from a first animal to be used as a feeder tissue;
maintaining the feeder tissue in contact with a culture medium;
contacting a pluripotent cell from a second animal with the feeder tissue;
incubating the pluripotent cell together with the feeder tissue in the culture medium; and
recovering a differentiated cell line derived from the pluripotent cell;
wherein the differentiated cell line is suitable for use in transplantation.

16. The method of claim 15, wherein the feeder tissue is cultured in vitro.

17. The method of claim 15, wherein the pluripotent cell is isolated from a human.

18. The method of claim 15, further comprising selecting feeder tissue derived from a dopaminergic neuron.

19. The method of claim 15, wherein the differentiated cell line is suitable for use in a transplantation of a nerve cell for treatment of a neurodegenerative disease.

20. The method of claim 19, wherein the neurodegenerative disease is Alzheimer's disease.
